(19) United States
(12) Patent Application Publication
(10) Pub. No.: US 2026/0026716 A1
(43) Pub. Date: Jan. 29, 2026

(54) INSERTION DEVICE

(71) Applicant: Roche Diabetes Care, Inc., Indianapolis, IN (US)

(72) Inventor: Harald Von Campenhausen, Ladenburg (DE)

(21) Appl. No.: 19/345,231

(22) Filed: Sep. 30, 2025

Related U.S. Application Data
(63) Continuation of application No. PCT/EP2024/060232, filed on Apr. 16, 2024.

(30) Foreign Application Priority Data
Apr. 19, 2023 (EP) 23168678.3

(51) Int. Cl. A61B 5/145; A61B 5/1473

(52) U.S. Cl. CPC A61B 5/14532; A61B 5/1473; A61B 2560/063

(57) ABSTRACT

An insertion device for inserting at least an insertable part of a medical device into a body tissue of a subject is proposed, that includes: at least one housing with at least one holding structure adapted to releasably receive the medical device, at least one insertion tool configured for inserting at least the insertable part of the medical device, at least one wheel comprising a guiding structure configured for guiding an axial movement of the insertion tool and the holding structure between a distal position and a proximal position, and at least one torsional drive configured for rotating the wheel upon activation, wherein the guiding structure comprises a shape with an eccentricity relative to a center of the wheel and/or relative to a turning point of the wheel, wherein the eccentricity is unequal to zero, and wherein the shape is configured to slow a linear movement around a turning point of the insertion tool.

### CROSS REFERENCE TO RELATED APPLICATIONS

This is a continuation of application serial no. PCT/EP2024/060232 filed Apr. 16, 2024 which claims priority from EP 23 168 678.3 filed Apr. 19, 2023 the entire disclosures of each of which are hereby incorporated herein by reference.

### BACKGROUND

### Technical Field

The present disclosure relates to an insertion device, an insertion system and to a method for inserting at least an insertable part of a medical device into a body tissue of a subject. The medical device may specifically be configured for detecting at least one analyte in a body fluid of the subject. The insertion device, the insertion system and the method may be applied in the field of continuous monitoring of the analyte in the body fluid of the subject, specifically in the field of home care and in the field of professional care, such as in hospitals. Other applications, however, are also feasible.

### Background Art

Monitoring certain body functions, more particularly monitoring one or more analyte concentrations such as one or more metabolite concentrations in a body fluid of a subject plays an important role in the prevention and treatment of various diseases. Such analytes can include by way of example, but not exclusively, glucose, lactate, cholesterol or other types of analytes and metabolites. Without restricting further possible applications, the disclosed embodiments will be described in the following text with reference to glucose monitoring. However, additionally or alternatively, the present disclosure can also be applied to other types of analytes.

Blood glucose monitoring, besides by using optical measurements, specifically may be performed by using electrochemical biosensors. In addition to so-called spot measurements, in which a sample of a body fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM) for example has been established as another important method for managing, monitoring and controlling a diabetes state.

In the process, an active sensor region is applied directly to a measurement site, which is generally arranged in an interstitial tissue, and, for example, converts glucose into electrical charge by using an enzyme (e.g. glucose oxidase, GOD), which charge is related to the glucose concentration and can be used as a measurement variable. Examples of such transcutaneous measurement systems are described in U.S. Pat. No. 6,360,888 B1 or in US 2008/0242962 A1.

Hence, current continuous monitoring systems typically are transcutaneous systems or subcutaneous systems. This means that the actual sensor or at least a measuring portion of the sensor is arranged under the skin of the user. However, an evaluation and control part of the system (also referred to as a patch) is generally situated outside of the body of the user, outside of the human or animal body. In the process, the sensor is generally applied using an insertion instrument, which is likewise described in U.S. Pat. No. 6,360,888 B1 in an exemplary fashion. Other types of insertion instruments are also known. Usually, a subcutaneous analyte sensor is based on a plastic substrate and, thus, cannot be inserted by itself and demands a metallic cannula. The insertion cannula typically possesses a special void for placing the sensor in it. During the insertion process, the cannula containing the sensor, penetrates the skin and retracts leaving the sensor inserted. One of the important roles of the insertion device may be moving the cannula with a well-defined initial speed, to successfully penetrate the skin without causing excessive pain. In order to achieve an initial momentum, the insertion mechanism usually possesses a mechanical assembly, which releases the cannula after some predefined force threshold is exceeded during the insertion process.

Inserters for invasive medical devices configured to insert a sensor or a cannula for drug delivery are commonly known in the prior art. They usually use one or two tension or compression springs to effect the insertion and retraction movement of the insertion cannula.

US 2015/0025338 A1 describes a device for applying an on-skin sensor assembly to skin of a host, the device comprising: a base configured to secure a housing, wherein the housing is configured to receive an electronics unit, wherein the electronics unit is configured to generate analyte information based on a signal from a sensor; a sensor insertion mechanism configured to insert the sensor into the host; and a trigger configured, in response to being activated, to cause the sensor insertion mechanism to insert the sensor into the host, to secure the electronics unit to the housing such that the sensor electrically contacts the electronics unit, and to cause the housing to detach from the base.

US 2013/0267811 A1 describes systems and methods for measuring an analyte in a host. More particularly, the described embodiments provide sensor applicators and methods of use with pushbutton activation that implant the sensor, withdraw the insertion needle, engage the transmitter with the housing, and disengage the applicator from the housing, all in one smooth motion. Some embodiments contemplate engagement of the transmitter with the housing after release of the applicator.

U.S. Pat. No. 11,400,215 B2 describes a medicament delivery device for delivery of medicament from a container into an injection site through a cannula. The device comprises a chassis, a drive mechanism for driving a piston member along a container axis to expel the medicament through the cannula, and a carriage for retaining the container, the cannula and the drive mechanism. An insertion mechanism is provided for moving the carriage in an insertion direction relative to the chassis, and a cannula bending mechanism is provided for bending the cannula about a bending axis to substantially align at least an end part of the cannula with the insertion direction prior to movement of the carriage in the insertion direction.

US 2009/0198215 A1 describes various embodiments which are directed to patches for medical devices. In various embodiments, an adhesive patch of a medical device may have selective areas with adhesive material of varying adhesion strengths. In other embodiments, an adhesive patch of a medical device may include adhesive material that may be activated by a catalyst to increase or decrease the adhesion strength of the adhesive material. In further embodiments, a medical device may include a pierceable membrane containing an agent, the pierceable membrane positioned to be pierced by a needle and to cause some of the agent to be carried to the user-patient.

EP 1 090 584 B1 describes a lancet unit for taking blood samples for diagnostic purposes which comprises a drive spring and a rotor which has a working surface with vertices at a maximum distance from its axis of rotation, and is connected to lancet holder in such a way that the lancet point emerges from the lancet opening while the rotor relative to the spring roller is within the angular thrust zone following each vertex.

U.S. Pat. No. 7,273,484 B2 describes a blood withdrawal system for withdrawing blood for diagnostic purposes. The blood withdrawal system comprises housing with a lancet, which can be moved within the housing. The housing also includes a lancet drive with a drive spring and a cocking mechanism that includes an actuation element. The blood withdrawal system of the present invention also includes a lancet ejection mechanism that can be moved from an active position to remove the lancet to a passive position where such removal of the lancet is not possible.

There still are several technical challenges are associated with the hitherto known devices. Specifically, known inserters usually require a technically complex design to enable safe and well-tuned insertion and withdrawal of the insertion cannula (with sensor or soft cannula). Therefore, there is a need for improved inserters.

### SUMMARY

It is desirable to provide an insertion device, an insertion system and a method for inserting at least an insertable part of a medical device into a body tissue of a subject which at least partially address the above-mentioned technical challenges. Such as an insertion device, an insertion system and a method for inserting at least an insertable part of a medical device into a body tissue of a subject are desirable which a) allow a reliable insertion process of at least the insertable part of the medical device into the body tissue of the subject, b) technically less complex in design and/or c) less expensive to manufacture than hitherto known devices.

At least some of these problems are solved by the provision of an insertion device, an insertion system and to a method for inserting at least an insertable part of a medical device into a body tissue of a subject with the features of the embodiments of the present disclosure. Advantageous embodiments which might be realized in an isolated fashion or in any arbitrary combinations are described throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, an insertion device for inserting at least an insertable part of a medical device into a body tissue of a subject is disclosed. The insertion device comprises:
at least one housing with at least one holding structure adapted to releasably receive the medical device,
at least one insertion tool configured for inserting at least the insertable part of the medical device,
at least one wheel comprising a guiding structure configured for guiding an axial movement of the insertion tool and the holding structure between a distal position and a proximal position, and
at least one torsional drive configured for rotating the wheel upon activation.

The guiding structure comprises a shape with an eccentricity relative to a center of the wheel and/or relative to a turning point of the wheel. The eccentricity is unequal to zero. The shape is configured to slow a linear movement around a turning point of the insertion tool.

Embodiments of the present disclosure may be associated with a much simpler design or architecture than hitherto known insertion systems, because there is no need to provide separate actuators or mechanisms to accomplish a controlled insertion and retraction movement of the insertion tool. As a result, manufacturing costs, processes and costs can be reduced.

The term “subject” as used herein is a broad term and is to be given its ordinary and customary meaning to a person of ordinary skill in the art and is not to be limited to a special or customized meaning. The term specifically relates to a human being or an animal, independent from the fact that the human being or animal, respectively, may be in a healthy condition or may suffer from one or more diseases. The subject may be a patient. As an example, the subject may be a human being or an animal suffering from diabetes. The subject may be a user, e.g. a patient, intending to monitor an analyte value, such as a glucose value, in the user's body tissue and/or to deliver medication, such as insulin, into the user's body tissue. However, in an embodiment, the user of the insertion device may be different from the subject. Additionally, or alternatively, the present disclosure may be applied to other types of users or patients.

The term “medical device” as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, exemplarily in the field of medical analytics or medical diagnostics. The medical device may be configured for performing at least one medical function and/or for being used in at least one medical process, such as one or more of a therapeutic process, a diagnostic process or another medical process.

The medical device may be configured to be mounted on a skin site, such as a skin site of an extremity of the subject. The extremity may be selected from the group consisting of: an arm, exemplarily an upper arm; a stomach; a shoulder; a back; hip; a leg. Exemplarily, the extremity may be the upper arm. However, also other applications may be feasible.

The medical device may comprise at least one component which may be configured to stay outside of the body tissue. Further, the medical device may comprise at least one insertable portion. The insertable portion may be configured for being inserted into the body tissue of the subject. Exemplarily, the insertion device may be configured for inserting the insertable portion of the medical device into the body tissue of the subject.

The medical device may comprise at least one device selected from the group consisting of: an analyte sensor device, e.g. an analyte sensor device configured for detecting at least one analyte in a body fluid of the subject; an infusion device, e.g. a drug infusion device configured for infusing a liquid drug such as insulin or any other liquid drug into a tissue of the subject; and an electrical stimulating device, e.g. configured for stimulating a body tissue of the subject. Such infusion devices include infusion pumps that can be attached to the skin of a patient also known as patch pumps or infusion sets that are connected to an infusion pump often worn in the pocket of a patient and connected to the infusion set by a tubing to deliver the drug from the pump into the skin. Known patch pumps include insulin pumps such as Roche's Accu-Chek® Solo Micropump or Terumo's Medisafe patch pump. Known infusion sets, in turn, include Roche's Accu-Chek® Insight Flex or Tandem's Diabetes AutoSoft Infusion set or the infusion sets manufactured by Convatec® such as Neria Guard.

For example, the medical device may comprise at least one analyte sensor device. The analyte sensor device may comprise at least one analyte sensor configured for detecting at least one analyte in a body fluid of the subject. The insertion tool of the insertion device may be configured for inserting at least a part of the analyte sensor into the body tissue of the subject, e.g. at least one insertable portion.

The term “analyte sensor device” as used herein is a broad term and is to be given its ordinary and customary meaning to a person of ordinary skill in the art and is not to be limited to a special or customized meaning. The term specifically may refer, without limitation, to a device configured for detecting at least one analyte in a body fluid of the subject.

The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the subject. Exemplarily, the analyte may be a metabolite or a combination of two or more metabolites. As an example, the analyte may be selected from the group consisting of: glucose, lactate, triglycerides, cholesterol. A preferred analyte is glucose. Still, other analytes or combinations of two or more analytes may be detected. The body tissue exemplarily may be or may comprise fatty tissue and/or interstitium. Other types of body tissue, however, are feasible.

The analyte sensor may be configured for being used in qualitatively and/or quantitatively detecting the at least one analyte. The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte.

The analyte sensor may be an electrochemical analyte sensor. The analyte sensor may comprise at least two electrodes. Specifically, the analyte sensor may comprise at least one two-electrode sensor. The two-electrode sensor may comprise precisely two electrodes, such as a working electrode and at least one further electrode such as a counter electrode, e.g. a working electrode and a combined counter/reference electrode. The working electrode may comprise a working electrode pad and, optionally, at least one test chemical disposed thereon. The counter electrode may comprise a counter electrode pad. Additionally and optionally, one or more redox materials may be disposed thereon. The analyte sensor may further comprise one or more leads for electrically contacting the electrodes. The leads may, during insertion or at a later point in time, be connected to one or more electronic components. For example, the leads may already be connected to the electronic components before insertion of the analyte sensor. For example, the analyte sensor may be a needle-shaped or a strip-shaped analyte sensor having a flexible substrate and the electrodes disposed thereon. As an example, the analyte sensor may have a total length of 5 mm to 50 mm, specifically a total length of 7 mm to 30 mm. The term “total length” within the context of the present invention relates to the overall length of the analyte sensor which means a portion of the analyte sensor which is inserted and the portion of the analyte sensor which may stay outside of the body tissue. The portion of the analyte sensor which is inserted is also called the in-vivo portion, the portion of the analyte sensor which may stay outside of the body tissue is also called the ex vivo portion. For example, the in vivo portion has a length in the range from 3 mm to 12 mm. The analyte sensor may further comprise a biocompatible cover, such as a biocompatible membrane which fully or partially covers the analyte sensor and which prevents the test chemical from migrating into the body tissue and which allows for a diffusion of the body fluid and/or the analyte to the electrodes. Other embodiments of electrochemical analyte sensors, such as three-electrode sensors, may be feasible. For example, the three-electrode sensor may comprise, in addition to the working electrode and the counter electrode, a reference electrode. Such analyte sensors are generally known in the art and include continuous glucose sensor systems such as Dexcom's G6 or G7 glucose sensor system, Medtronic's Enlite Glucose sensor or Abbott's Freestyle Libre 2 or 3.

The analyte sensor may be an optical analyte sensor. For example, the analyte sensor may comprise a flexible light guide with glucose sensitive coating at its end and/or a tube-like carrier with functional elements at inner or outer walls. Other embodiments of the analyte sensor may be possible too.

The term “body fluid” as used herein is a broad term and is to be given its ordinary and customary meaning to a person of ordinary skill in the art and is not to be limited to a special or customized meaning. The term specifically relates to an arbitrary fluid which typically is present in a body or body tissue of a user or a patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of at least one analyte, the body fluid may be present within the body or body tissue.

The medical device may comprise at least one electronics unit. The analyte sensor may be operably connected to the electronics unit. The term “electronics unit” as used herein is a broad term and is to be given its ordinary and customary meaning to a person of ordinary skill in the art and is not to be limited to a special or customized meaning. The term specifically may refer, without limitation, to an arbitrary unit, such as a unit which may be handled as a single piece, which is configured for performing at least one electronic function. For example, in case the medical device is the medical device for detecting the analyte in the body fluid of the user, the electronics unit may have at least one interface for being connected to the analyte sensor, wherein the electronics unit may provide at least one electronic function interacting with the analyte sensor, such as at least one measurement function. The electronics unit may be configured for one or more of determining and/or controlling a detection of the analyte and/or transmitting measurement data to another component. Specifically, the electronics unit may be configured for one or more of performing a measurement with the analyte sensor, performing a voltage measurement, performing a current measurement, recording sensor signals, storing measurement signals and/or measurement data, transmitting sensor signals to another component. Thus, the electronics unit specifically may comprise at least one of: a voltmeter, an ammeter, a potentiostat, a voltage source, a current source, a signal receiver, a signal transmitter, an analog-digital converter, an electronic filter, a data storage device, an energy storage. For example, the electronics unit may be embodied as a transmitter or may comprise at least one transmitter configured for transmitting data to a remote computer or to a remote device.

The medical device further may comprise at least one electronic remote device configured to communicate and/or control with the medical device. The electronic remote device may be selected from a personal computer, a wearable, a smartphone, a proprietary remote control, a tablet or a server.

For example, the medical device may comprise at least one infusion device. The term “infusion device” as used herein is a broad term and is to be given its ordinary and customary meaning to a person of ordinary skill in the art and is not to be limited to a special or customized meaning. The term specifically relates to a device configured for delivering and/or infusing at least one medication into the body tissue of the user, e.g. for delivering and/or infusing insulin into the body tissue of the user. The infusion device may be a drug infusion device. The infusion device may comprise at least one infusion cannula. The insertion tool of the insertion device may be configured for inserting at least a part of the infusion cannula into the body tissue of the subject. The term “infusion cannula” as used herein is a broad term and is to be given its ordinary and customary meaning to a person of ordinary skill in the art and is not to be limited to a special or customized meaning. The term exemplarily may refer, without limitation, to a hollow tube configured for delivering and/or infusing a medication into the body tissue of the subject, e.g. for delivering and/or infusing insulin into the body tissue of the subject.

For example, the medical device may comprise at least one electrical stimulation device. The term “electrical stimulation device” as used herein is a broad term and is to be given its ordinary and customary meaning to a person of ordinary skill in the art and is not to be limited to a special or customized meaning. The term specifically relates to a device configured for applying at least one electrical stimulation to the body tissue. The electrical stimulation device may comprise a stimulation electrode. The insertion tool may be configured for inserting at least a part of the stimulation electrode into the body tissue of the subject.

The term “inserting” as used herein is a broad term and is to be given its ordinary and customary meaning to a person of ordinary skill in the art and is not to be limited to a special or customized meaning. The term specifically may refer, without limitation, to an action or process of one or more of transcutaneously or subcutaneously implanting and/or positioning the medical device, exemplarily the insertable portion of the medical device, into the body tissue of the subject. The medical device may partially be inserted into the body tissue. The insertion of the medical device may be performed by using the insertion device. After insertion, at least an insertable part of the medical device may remain in the body tissue of the subject for a predetermined period of time, such as for several hours, exemplarily for one or more days, such as for up to one week, or such as for up to two weeks or even more.

The term “insertion device” as used herein is a broad term and is to be given its ordinary and customary meaning to a person of ordinary skill in the art and is not to be limited to a special or customized meaning. The term specifically relates to a device configured for inserting at least the insertable part of the medical device into the body tissue. The insertion device may be configured for transcutaneously or subcutaneously inserting at least the insertable part of the medical device into the body tissue, such as by performing an incision or a puncture in a skin of the subject and by transferring the medical device partially into the body tissue. The insertion device may be removed fully or partially after inserting the medical device at least partially into the body tissue of the subject.

As outlined above, the insertion device comprises the at least one housing with the at least one holding structure adapted to releasably receive the medical device.

The term “housing” as used herein is a broad term and is to be given its ordinary and customary meaning to a person of ordinary skill in the art and is not to be limited to a special or customized meaning. The term specifically relates to an element or component having at least one interior space and at least one wall fully or partially surrounding the at least one interior space and providing protection to the interior space, such as one or more of a mechanical protection and a protection against environmental influences such as one or more of moisture, oxygen and microbial contaminations. The housing may generally be adapted to fully or partially surround and/or receive one or more elements in order to provide one or more of a mechanical protection, a mechanical stability, an environmental protection against moisture and/or ambient atmosphere, a shielding against electromagnetic influences or the like. The housing may also provide a basis for attachment and/or holding one or more further components or elements.

The term “holding structure” as used herein is a broad term and is to be given its ordinary and customary meaning to a person of ordinary skill in the art and is not to be limited to a special or customized meaning. The term specifically relates to an arbitrary element configured for at least partially enclosing at least one element, e.g. the medical device and/or the insertion tool, and/or for at least partially holding the at least one element in a desired position. The holding structure may be configured for holding the medical device, i.e. a medical device holder, and/or the holding structure may be configured for holding the insertion tool, i.e. an insertion tool holder. For example, the holding structure may be configured for holding the insertion tool and may serve as a medical device holder. Alternatively, the insertion tool holder and medical device holder may be two separate elements.

The holding structure, as will be outlined in more detail below, may be or may comprise at least one plunger or may be connected to at least one plunger in order to drive the insertion tool to perform a puncture or insertion motion into the body tissue for inserting the medical device and back.

Exemplarily, the holding structure may comprise at least one receptacle and the medical device may be at least partially received in the receptacle. For example, the holding structure may comprise a rotational symmetric hollow center. The rotational symmetric hollow center may exemplarily be open to a proximal end of the holding structure. The medical device may be arrangeable at the proximal end of the holding structure. The holding structure may comprise one or more elements configured for engaging with the medical device, exemplarily with a surface of the medical device, for the purpose of supporting the medical device and keeping the medical device in position. The holding structure may comprise one or more of at least one finger, at least one gripper, at least one hook, at least one pincer or the like. The elements may be arranged at an inner structure of the holding structure, exemplarily at an inner structure of the receptacle of the holding structure. Also, other embodiments may be feasible. Exemplarily, the holding structure may comprise at least one adhesive element. The elements may be arranged at an inner structure of the holding structure, exemplarily at an inner structure of the receptacle of the holding structure.

The term “releasably receiving the medical device” as used herein is a broad term and is to be given its ordinary and customary meaning to a person of ordinary skill in the art and is not to be limited to a special or customized meaning. The term specifically relates to the fact that a connection between the holding structure and the medical device is at least one of separable, detachable, and non-permanent. The holding structure may be configured for directly or indirectly withdrawing the insertion tool from the medical device while leaving at least the inserted part of the medical device in the body tissue. The term “directly withdrawing” as used herein is a broad term and is to be given its ordinary and customary meaning to a person of ordinary skill in the art and is not to be limited to a special or customized meaning. The term specifically relates to embodiments in which the holding structure is configured for applying at least one force to the medical device thereby releasing and/or disconnecting the holding structure and the medical device. The term “indirectly withdrawing” as used herein is a broad term and is to be given its ordinary and customary meaning to a person of ordinary skill in the art and is not to be limited to a special or customized meaning. The term specifically relates to embodiments in which at least one force is applied to the holding structure and/or a further component of the insertion device thereby releasing and/or disconnecting the holding structure and the medical device.

The holding structure may be configured for directly or indirectly advancing the holding structure and at least the insertable part of the medical device axially towards the body tissue and for inserting the insertion tool and at least the insertable part of the medical device into the body tissue. The term “advancing” as used herein is a broad term and is to be given its ordinary and customary meaning to a person of ordinary skill in the art and is not to be limited to a special or customized meaning. The term specifically relates to accelerating, e.g. from rest to a defined speed. The term “directly advancing” as used herein is a broad term and is to be given its ordinary and customary meaning to a person of ordinary skill in the art and is not to be limited to a special or customized meaning. The term specifically relates to embodiments in which the holding structure is configured for applying at least one force to the medical device thereby advancing the medical device. The term “indirectly advancing” as used herein is a broad term and is to be given its ordinary and customary meaning to a person of ordinary skill in the art and is not to be limited to a special or customized meaning. The term specifically relates to embodiments in which at least one force is applied to the holding structure and/or a further component of the insertion device thereby advancing the medical device.

The term “axial movement” as used herein is a broad term and is to be given its ordinary and customary meaning to a person of ordinary skill in the art and is not to be limited to a special or customized meaning. The term specifically relates to a movement in axial direction. An axial direction may be defined by an axis of extension of the insertion device, e.g. of the housing. Specifically, the term means a movement towards or away from the skin, specifically a movement essentially perpendicular to the surface of the skin where the insertion device can be attached to.

The insertion device may comprise at least one insertion locking mechanism. The insertion locking mechanism may be configured for locking and releasing movement of the holding structure. The insertion locking mechanism may be configured for interacting with one or more of the wheel, the holding structure guide or the medical device. For example, advancing the holding structure may be triggered by releasing the insertion locking mechanism. The term “insertion locking mechanism” as used herein is a broad term and is to be given its ordinary and customary meaning to a person of ordinary skill in the art and is not to be limited to a special or customized meaning. The term specifically relates to a device or combination of devices adapted for performing at least one locking function. The insertion locking mechanism may be at least one mechanism configured for locking and/or releasing movement of the holding structure, e.g. a plunger of the holding structure, by e.g. interacting with one or more of the wheel, a holding structure guide, or the medical device.

For example, the insertion locking mechanism may be adapted for preventing an unwanted actuation of the insertion device, thereby preventing an unwanted driving of the holding structure. As an example, the holding structure is supported within the housing in a movable fashion, such as by providing one or more bearings or one or more guides, such as one or more guide rails or slides. For example, the holding structure may be stored or guided in a linearly movable fashion within the housing of the insertion device. The holding structure may have a retracted position or a rest position, in which the holding structure is stored before insertion and in which the insertion tool does not protrude from the insertion device. The insertion locking mechanism may be adapted to prevent the holding structure from unwantedly leaving the rest position or a storage position and/or may be adapted to generally prevent an unwanted firing of the insertion device. The term “firing” as used herein is a broad term and is to be given its ordinary and customary meaning to a person of ordinary skill in the art and is not to be limited to a special or customized meaning. The term specifically relates to an actuation of the insertion device and/or a process of movement towards the distal position of the holding structure. The insertion locking mechanism may comprise at least one locked position and at least one unlocked position, wherein in the locked position, an unwanted firing may be blocked, and wherein in the unlocked position, a firing of the insertion device and, thus, an insertion of the insertable portion of the medical device into the body tissue, is enabled. For example, the insertion locking mechanism, in a locked position, is adapted to at least partially block a rotation of the wheel and/or movement of the holding structure. The insertion locking mechanism further may act as a transportation lock or as a transportation safety device, such as for preventing an unwanted firing of the insertion device during transportation or storage. Still, an additional transportation locking device or transportation safety device may be implemented additionally.

The insertion locking mechanism may comprise one or more blocking elements. The blocking element may abut on one or more elements of the insertion device, e.g. the wheel and/or the holding structure in the locked position. The one or more blocking elements may be movable and may, in the locked position, directly or indirectly abut the wheel or a part thereof in order to prevent a rotation of the wheel and/or movement of the holding structure. In the unlocked position, the one or more blocking elements may be moved out of the way of the wheel and/or the holding structure in order to free the wheel and/or the holding structure and in order to permit the rotation of the wheel and/or movement of the holding structure. For example, the blocking element may be at least one locking arm configured for preventing movement of the holding structure from the distal position to the proximal position.

The term “distal position” as used herein is a broad term and is to be given its ordinary and customary meaning to a person of ordinary skill in the art and is not to be limited to a special or customized meaning. The term specifically relates to a position specification indicating a position of the insertion device and/or any parts thereof and/or of the medical device in relation to the subject in which the insertion tool, the holding structure and/or the medical device are furthermost from the proximal side of the insertion device. Exemplarily, for inserting at least the insertable part of the medical device, the insertion device may be brought into contact with a skin site of the subject. The distal position may refer to a position being distanced to the skin site of the subject. The distal position may be an initial position prior to the insertion movement of the insertion device and/or any parts thereof. Each component of the insertion device may have its own and/or individual distal position. For example, the insertion tool, the holding structure and/or the medical device may have their own and/or individual distal positions, respectively. Prior to insertion, the insertion tool, the holding structure and/or the medical device may be in their distal position and may be ready for inserting at least the insertable part of the medical device into the body tissue of the subject.

The term “proximal position” as used herein is a broad term and is to be given its ordinary and customary meaning to a person of ordinary skill in the art and is not to be limited to a special or customized meaning. The term specifically relates to a position specification indicating a position of the insertion device and/or any parts thereof and/or of the medical device in relation to the subject in which the insertion tool, the holding structure and/or the medical device are closest to the proximal side of the insertion device. Exemplarily, for inserting the medical device, the insertion device may be brought into contact with the skin site of the subject. The proximal position may refer to a position being in close proximity to the skin site of the subject. Each component of the insertion device may have its own and/or individual proximal position. For example, the insertion tool, the holding structure and/or the medical device may have their own and/or individual proximal positions, respectively. In case the insertion tool, the holding structure and/or the medical device are in their proximal position, the medical device may be inserted into the body tissue of the subject. During insertion of the medical device, the medical device may be in contact with the skin site of the subject and, thus, may be in its proximal position.

The insertion locking mechanism may be operable by hand. For operating the insertion locking mechanism, such as triggering releasing the insertion locking mechanism, the insertion locking mechanism may comprise at least one operation element such as at least one switch, e.g. at least one slide switch, a button to push, a release to pull, at least one removeable pin and the like. However, other types of operation elements are feasible.

For example, the holding structure may comprise at least one plunger. The plunger may also be referred to as a piston, a piston rod or a push rod. The plunger may be configured for directly or indirectly, e.g. via at least one further element, attaching to the medical device and/or the insertion tool. The plunger may be configured to directly or indirectly advance the medical device axially towards the skin and to insert the insertion tool, e.g. an insertion cannula, into the skin in a first phase. The plunger may be configured to withdraw the insertion tool from the medical device while leaving the inserted part of the medical device in the skin in a second phase.

The holding structure may comprise at least one holding structure guide, e.g. a guide for the plunger. The term “holding structure guide” as used herein is a broad term and is to be given its ordinary and customary meaning to a person of ordinary skill in the art and is not to be limited to a special or customized meaning. The term specifically relates to at least one element configured for guiding a movement of the holding structure. The guiding may comprise directing the motion of the holding structure. For example, the housing may comprise at least one guiding frame configured for guiding an axial movement of the holding structure guide between its distal position and its proximal position. The insertion locking mechanism may comprise at least one locking arm configured for engaging with the holding structure guide for preventing movement of the holding structure from the distal position to the proximal position. The at least one holding structure guide may specifically be present at the interface of the holding structure guide with the guiding frame which at least one holding structure guide is configured to guide the axial movement of the holding structure in contact with the corresponding guiding structure of the guiding frame. Examples of guiding elements include rails, channels tongues, grooves, keys and slots.

As outlined above, the insertion device comprises the at least one insertion tool configured for inserting at least an insertable part of the medical device. The term “insertion tool” as used herein is a broad term and is to be given its ordinary and customary meaning to a person of ordinary skill in the art and is not to be limited to a special or customized meaning. The term specifically relates to an arbitrary element which may be insertable at least partially into the body tissue, particularly in order to deliver or to transfer a further element. The insertion tool may be configured for supporting the insertion of the insertable part of the medical device. Exemplarily, the insertion tool may be configured for supporting the insertion of the insertable portion of the medical device. The insertion tool may comprise a tip or a sharp end for inserting the insertable part of the medical device, exemplarily the insertable portion of the medical device, into the body tissue. In the context of the present invention, the insertion tool is considered as part of the insertion device. However, in particular during manufacturing of an insertion system the insertion tool may also be considered as an insertable part of the medical device. Thus, during insertion, the insertion tool may perform a puncture motion from the distal to the proximal position, thereby creating an incision in a skin of a user or patient, transferring at least the insertable part of the medical device into the body tissue, and, subsequently, a motion into the reverse direction, wherein the insertion tool is pulled out from the body tissue, wherein at least the insertable part of the medical device at least partially remains within the body tissue. During insertion, at least the insertable part of the medical device may fully or partially be surrounded by the insertion tool. The holding structure, as outlined above, may be or may comprise at least one plunger which is adapted for pushing the insertion tool axially to perform the puncture motion and which may further be adapted to axially pull back the insertion tool after insertion. After insertion, the insertable part of the medical device may remain in the body tissue of the subject. The insertion tool, however, may be retracted from the body tissue of the subject into the insertion device after inserting the medical device.

The insertion tool may comprise at least one insertion cannula or at least one insertion needle. The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially slotted such as in U shape, triangular shape, ellipsoid shape or round shape. At least the insertable part of the medical device may be received within the insertion cannula, such as within a lumen of the insertion cannula. The term “insertion needle” as used herein is a broad term and is to be given its ordinary and customary meaning to a person of ordinary skill in the art and is not to be limited to a special or customized meaning. The term specifically may refer, without limitation, to a compact needle, exemplarily without a slot and without any hollow parts. The medical device such as a soft cannula of an infusion set of a patch pump may be received on an outer surface of the insertion needle.

The insertion device may comprise an insertion tool protection mechanism, e.g. an insertion cannula or insertion needle protection mechanism, configured for blocking movement of the holding structure towards the proximal position again. Using an insertion cannula or insertion needle protection mechanism may protect the subject from getting into contact with an exposed insertion tool. For example, the insertion locking mechanism may be used as insertion cannula or insertion needle protection mechanism. Using the same mechanism may allow simplifying the design. Alternatively, a different mechanism from the insertion locking mechanism may be used to block the movement of the holding structure. For example, the insertion device may be configured for a locking of the holding structure after retraction. A number of safety measures may be feasible to secure the retracted insertion tool inside the housing. For example, a torsional spring, which is described in more detail below, may be locked and/or uncoupled from the wheel, e.g. once the holding structure reaches its distal position. For example, the holding structure, e.g. the plunger, and/or the insertion tool, may be locked e.g. once the holding structure reaches its distal position.

As outlined above, the insertion device comprises the at least one wheel comprising a guiding structure configured for guiding an axial movement of the insertion tool and the holding structure between a distal position and a proximal position.

The term “wheel” as used herein is a broad term and is to be given its ordinary and customary meaning to a person of ordinary skill in the art and is not to be limited to a special or customized meaning. The term specifically relates to at least one element configured for exerting a rotational movement about an axis or a center. For example, the shape of the wheel may be selected from the group consisting of circular, elliptic, non-circular, triangular, rectangular or polygonal. The wheel may be a flywheel. The wheel may be a cam or a face cam.

The term “guiding structure” as used herein is a broad term and is to be given its ordinary and customary meaning to a person of ordinary skill in the art and is not to be limited to a special or customized meaning. The term specifically may refer, without limitation, to at least one two- or three-dimensional structure or plurality of structures configured for directing the movement, e.g. defining a velocity and/or acceleration of the axial movement, of the insertion tool and the holding structure. The guiding structure may be a guiding rail and/or a guiding channel. The term “guiding channel” as used herein is a broad term and is to be given its ordinary and customary meaning to a person of ordinary skill in the art and is not to be limited to a special or customized meaning. The term specifically may refer, without limitation, to a groove within a surface plane of the wheel. The term “guiding rail” as used herein is a broad term and is to be given its ordinary and customary meaning to a person of ordinary skill in the art and is not to be limited to a special or customized meaning. The term specifically may also refer, without limitation, to a protrusion extending from the surface plane of the wheel.

The guiding structure comprises a shape with an eccentricity relative to a center of the wheel and/or relative to a turning point of the wheel. The eccentricity is unequal to zero.

For example, the shape of the guiding structure, e.g. in a plan view, is selected from the group consisting of non-circular, elliptic, spiral-shaped, and triangular. For example, the shape of the guiding structure may be circular. Alternatively, the shape can be configured such that despite an essentially steady angular movement speed of the wheel, the time that the holding structure remains in an extended position, such as the proximal position, is longer than if the guiding structure has a strictly circular form with zero eccentricity. Such a non-circular shape can allow increasing the time of keeping the holding structure in the extended position. This design is associated with an advantage compared to hitherto known inserters that operate with high velocity insertion and retraction essentially throughout the whole insertion process: the insertion according to the present invention is able to provide more time for the insertable portion of the medical device to penetrate deeper into the skin before the insertion tool is pulled back: during insertion of the insertable portion of the medical device into the skin, due to the inertia of skin the area of the skin adjacent to the insertion point will move away from the medical device, in particular if the insertion occurs at a high axial movement velocity. When the speed of axial movement of the insertion tool around its point of closest proximity to the skin is slowed down, there is more time for the depressed part of the skin around the insertion point to relax and move back upwards towards the medical device. This way the insertion of the insertable portion of the medical device is improved because the insertable portion will penetrate the skin deeper before the axial retraction movement accelerates again to pull out the insertion tool from the skin while leaving the insertable portion in place.

In other embodiments also circular shapes are possible, e.g. in case of using a non-circular wheel and/or a non-central mounting point.

The term “center of the wheel” as used herein is a broad term and is to be given its ordinary and customary meaning to a person of ordinary skill in the art and is not to be limited to a special or customized meaning. The term specifically may refer, without limitation, to a mounting point of the wheel. The center may be a center of gravity. However, other mounting points are possible. The term “turning point of the wheel” as used herein is a broad term and is to be given its ordinary and customary meaning to a person of ordinary skill in the art and is not to be limited to a special or customized meaning. The term specifically may refer, without limitation, to a point around which the rotation of the wheel takes place. The wheel may have several turning points, e.g. for different times during movement. The shape is configured to slow a linear movement around a turning point of the insertion tool. The term “turning point of the insertion tool” as used herein is a broad term and is to be given its ordinary and customary meaning to a person of ordinary skill in the art and is not to be limited to a special or customized meaning. The term specifically may refer, without limitation, to a point at which the movement of the insertion tool from the distal to the proximal position is reversed.

The insertion device, e.g. the wheel, may comprise at least one follower. The follower may be attached to the holding structure. The guiding structure may be configured for guiding the follower. The term “follower” as used herein is a broad term and is to be given its ordinary and customary meaning to a person of ordinary skill in the art and is not to be limited to a special or customized meaning. The term specifically may refer, without limitation, to an arbitrary shaped element configured for engaging with the guiding structure and for moving along the guiding structure. For example, the follower may be a follower pin. The follower pin may move within the guiding channel. The guiding structure may guide the follower pin. The follower may be attached to the holding structure. For example, the follower may be a sleigh or wagon moving on the guiding rail. The sleigh or wagon may be attached to the holding structure. The shape of the guiding structure may be such that friction with the follower is increased in at last one angular position or section.

As outlined above, the insertion device comprises the at least one torsional drive configured for rotating the wheel upon activation. The term “torsional drive” as used herein is a broad term and is to be given its ordinary and customary meaning to a person of ordinary skill in the art and is not to be limited to a special or customized meaning. The term specifically relates to a driving mechanism working by torsion. For example, the torsional drive comprises at least one torsional spring. For example, when loading the torsional spring, the torsional spring is twisted, and can exert, upon activation, a torque in the opposite direction, thereby exerting a rotational force on the wheel. The wheel is configured for rotating in response to a rotational force exerted by the torsional drive. The torsional drive, e.g. the torsional spring, may be preload, e.g. ex-factory. The torsional drive, e.g. the torsional spring may be loadable by the user, e.g. the torsional drive may be reloadable. The term “rotating the wheel upon activation” as used herein is a broad term and is to be given its ordinary and customary meaning to a person of ordinary skill in the art and is not to be limited to a special or customized meaning. The term specifically relates to the fact that the torsional force exerts a rotational force to the wheel thereby triggering and/or driving a rotation of the wheel.

The insertion device may comprise at least one protective cap. The term “protective cap” as used herein is a broad term and is to be given its ordinary and customary meaning to a person of ordinary skill in the art and is not to be limited to a special or customized meaning. The term specifically may refer, without limitation, to a part of the housing configured for protecting the medical device and/or other components of the insertion device before use such as during storage, supply, sale and the like. One or more of the housing or the protective cap may comprise at least one desiccant. Such protective caps and desiccants in the sense of the present invention are further described in EP3727130A1 and EP3202323A1. The protective cap may be configured for covering a skin facing opening of the housing. The protective cap may be configured for providing an originality closure of the insertion device. For example, removal of the protective cap may release and/or unblocks the insertion locking mechanism.

The protective cap may be detachable from the insertion device by at least one twist movement. The protective cap, as an example, may be connected to the housing by at least one of a form-fit or a force-fit connection. The protective cap may be attached to the housing by one or more of friction contact, threads, and the like. A rim of the protective cap may be pushed over a rim of the housing or vice a versa. Thus, as an example, the protective cap may have a circular, oval or polygonal rim which fits tightly over the rim of the housing having a corresponding shape, or vice a versa. There may be an overlap region in the connected state, in which the protective cap overlaps with the housing or vice a versa.

The protective cap specifically may be fully or partially made of at least one rigid material, such as of at least one plastic material and/or at least one metal. The protective cap specifically may have an opening which is configured to be directed towards the housing of the insertion device. The protective cap specifically may be made essentially rotationally symmetric, e.g. by having an axial rotational symmetry about an axis such as a cylinder axis. The protective cap, as an example, may be designed as a cylinder, a hemi-sphere or as a dome.

For example, the torsional drive may be pre-tensioned ex-factory or by the user, preferably by pulling off the protective cap which in turn tensions the torsional drive. For example, the protective cap is connected to the rotation axis by a filament which turns the axis in an orientation that tensions the torsional spring when removing the protective cap from the housing. A ratchet mechanism at the rotation axis may be used to ensure that the tensioning of the rotation axis remains after the filament is pulled off from the rotation axis.

The insertion device may comprise a friction brake. The term “friction brake” as used herein is a broad term and is to be given its ordinary and customary meaning to a person of ordinary skill in the art and is not to be limited to a special or customized meaning. The term specifically may refer, without limitation, to a brake operating by friction. The friction brake may be is active in the proximal position of the insertion tool and the holding structure. Additionally or alternatively, a friction resistance of the friction brake is increased relative to other angular positions or sections of the guiding structure. The friction brake may be configured for interacting with the wheel and/or the holding structure. For example, the guiding structure may be shaped in a way that it enables to change the friction resistance between the guiding structure and the follower pin. This resistance, in turn, can be used to controlling the speed of movement of the wheel and as a result the speed of movement of the insertion and retractions movement of the holding structure. Alternatively or in combination with changing shape of the guiding structure the frictional resistance may be controlled by one or more of the material, texture of the surface or the coating used in different parts of the guiding structure.

The insertion device comprises at least one brake element. In the proximal position of the holding structure, e.g. the maximum extended position of the plunger, at least one part of the wheel may contact the brake element. The term “brake element” as used herein is a broad term and is to be given its ordinary and customary meaning to a person of ordinary skill in the art and is not to be limited to a special or customized meaning. The term specifically may refer, without limitation, to at least one element configured for reducing a velocity of the rotation, e.g. angular movement speed, of the wheel. The brake element may be configured for slowing down the angular movement speed of the wheel for a defined angular braking interval of the wheel. Once the end of the braking interval is reached the brake element may cease to inhibit wheel movement and the wheel may accelerate to retract the insertion tool. For example, the insertion device may be configured such that the plunger stops movement or moves with very low speed for at least one limited angular interval of wheel movement upon reaching the maximum extended position before initiating the retraction movement. For example, a wheel diameter may be larger in an angular interval where the braking occurs. In this aspect of the wheel an outer margin may contacts the brake element which results in slowing down the movement of the wheel. The brake element may comprise a bar, e.g. with a surface of high resistance such as rubber. Additionally or alternatively of extending the diameter of the wheel in a certain angular interval, it may be possible to locally increase the thickness of the wheel such that the brake element contacts the wheel only in the area of increased thickness. Additionally or alternatively, the wheel may comprises the follower pin which extends radially outwards from the wheel and which contacts the brake element such as a leaf spring. Alternatively, the follower pin can be mounted on the side of the wheel and be oriented perpendicular from the circular plane of the wheel. The control of the speed of axial movement of the insertion tool and the holding structure by the brake element can also be combined with control provided by the frictional brake.

The insertion device may comprise a medical device locking mechanism. The term “medical device locking mechanism” as used herein is a broad term and is to be given its ordinary and customary meaning to a person of ordinary skill in the art and is not to be limited to a special or customized meaning. The term specifically may refer, without limitation, to at least one element and/or a plurality of elements configured for preventing the medical device from being pulled back into the housing when the holding structure withdraws the insertion tool from the medical device. For example, the medical device locking mechanism may comprise bendable locking arms configured for preventing the medical device from being pulled back into the housing when the holding structure withdraws the insertion tool from the medical device.

The insertion device may comprise at least one user interface. The term “user interface” as used herein is a broad term and is to be given its ordinary and customary meaning to a person of ordinary skill in the art and is not to be limited to a special or customized meaning. The term may refer, without limitation, to an element or device which is configured for interacting with its environment, such as for the purpose of unidirectionally or bidirectionally exchanging information, such as for exchange of one or more of data or commands. For example, the user interface may be configured to share information with a user and to receive information by the user. The user interface may be a feature to interact visually with a user, such as a display, or a feature to interact acoustically with the user. The user interface, as an example, may comprise a sound generator configured for playing at least one sound when the insertion has been successfully completed. Specifically, the sound generator is also configured to play a melody, such as Celebration by Kool & the Gang.

In a further aspect of the present disclosure, an insertion system comprises at least one medical device and at least one insertion device, e.g. as described in a first aspect above or as descried in more detail below, for at least partially inserting the medical device into a body tissue of a subject. The medical device is held by the holding structure. For possible definitions and options, reference may be made to the disclosure of the insertion device described herein.

The term “system” as used herein is a broad term and is to be given its ordinary and customary meaning to a person of ordinary skill in the art and is not to be limited to a special or customized meaning. The term specifically may refer, without limitation, to a group of at least two elements which may interact with each other in order to fulfill at least one common function. The at least two components may be handled independently or may be coupled, connectable or integrable in order to form a common device.

The term “insertion system” as used herein is a broad term and is to be given its ordinary and customary meaning to a person of ordinary skill in the art and is not to be limited to a special or customized meaning. The term specifically may refer, without limitation, to a group of at least two elements or components which are capable of interacting with each other in order to perform at least one transcutaneous or subcutaneous insertion of at least one of the two elements or components into a body tissue of a subject, such as by performing an incision or a puncture in a skin of the subject and by transferring the at least one of the two elements or components fully or partially into the body tissue.

In a further aspect of the present disclosure, a method for inserting at least an insertable part of a medical device into a body tissue of a subject is disclosed. The method comprises using the insertion system as described above or as will further be described below in more detail. Thus, for possible definitions and options, reference may be made to the disclosure of the insertion system and the insertion device described herein.

The method comprises the following steps which specifically may be performed in the given order. The method may comprise further method steps which are not listed.

The method comprises the following steps:
a) applying the insertion system to a subject's skin;
b) inserting at least an insertable part of the medical device into the body tissue of the subject wherein the wheel is rotated upon activation by the at least one torsional drive, wherein an axial movement of the insertion tool and the holding structure between the distal position and the proximal position is driven by the wheel, and wherein the guiding structure comprises a shape with an eccentricity relative to a center of the wheel and/or relative to a turning point of the wheel, wherein the eccentricity is unequal to zero, and wherein the shape slows a linear movement around a turning point of the insertion tool.

The method further may comprise retracting the insertion tool from the body tissue of the subject into the insertion device after inserting the medical device.

The method may also be an in vivo method. Moreover, the method may comprise steps in addition to those explicitly mentioned above. For example, further steps may relate, e.g., to disinfecting the skin site before insertion. As is understood by the skilled person, the method does not require specific medical skills and does not impose a significant health risk on a subject and, thus, is usually performed by the subject receiving said medical device itself. Thus, the method may be a method of self-administration.

The method and devices according to the present invention provide a large number of advantages over known methods and devices. The disclosed drive mechanism may comprise a restricted guidance of insertion and retraction movement via the torsional drive driven wheel a guiding structure, e.g. a rail guide or a lead groove. The guiding structure may comprise a non-circular form decreasing a velocity of a movement at the end of an insertion process and delaying a retraction process. A course of the rail guide, together with a force of the torsional drive may determine a speed of the insertion and retraction movement and may make it possible, in particular, to decelerate the insertion tool's movement when the insertion tool is about to change a direction of movement. A delay of the retraction movement may support preventing an incomplete insertion of the analyte sensor when the insertion tool is pulled out of the body tissue. Thus, the insertion device according to the present invention may significantly improve a reliability of an insertion process. An incomplete insertion of the analyte sensor may be prevented by decreasing a velocity of a linear movement at the turning point of the wheel. By using the same torsional drive for the insertion and retraction movement of the holding structure the construction may be simple and robust in use.

Summarizing and without excluding further possible embodiments, the following embodiments may be envisaged:
Embodiment 1. An insertion device for inserting at least an insertable part of a medical device into a body tissue of a subject comprising:
at least one housing with at least one holding structure adapted to releasably receive the medical device,
at least one insertion tool configured for inserting at least the insertable part of the medical device,
at least one wheel comprising a guiding structure configured for guiding an axial movement of the insertion tool and the holding structure between a distal position and a proximal position, and
at least one torsional drive configured for rotating the wheel upon activation,
wherein the guiding structure comprises a shape with an eccentricity relative to a center of the wheel and/or relative to a turning point of the wheel, wherein the eccentricity is unequal to zero, and wherein the shape is configured to slow a linear movement around a turning point of the insertion tool.
Embodiment 2. The insertion device according to the preceding embodiment, wherein the holding structure is configured for directly or indirectly withdrawing the insertion tool from the medical device while leaving at least the inserted part of the medical device in the body tissue.
Embodiment 3. The insertion device according to any one of the preceding embodiments, wherein the holding structure is configured for directly or indirectly advancing the holding structure and at least the insertable part of the medical device axially towards the body tissue and for inserting the insertion tool and at least the insertable part of the medical device into the body tissue.
Embodiment 4. The insertion device according to the preceding embodiment, wherein advancing the holding structure is triggered by releasing an insertion locking mechanism.
Embodiment 5. The insertion device according to any one of the preceding embodiments, wherein the holding structure comprises at least one holding structure guide, wherein the housing comprises at least one guiding frame configured for guiding an axial movement of the holding structure guide between its distal position and its proximal position.
Embodiment 6. The insertion device according to any one of the two preceding embodiments, wherein the insertion locking mechanism comprises at least one locking arm configured for engaging with the holding structure guide for preventing movement of the holding structure from the distal position to the proximal position.
Embodiment 7. The insertion device according to any one of the preceding embodiments, wherein the holding structure comprises at least one plunger.
Embodiment 8. The insertion device according to any one of the preceding embodiments, wherein the wheel is configured for rotating in response to a rotational force exerted by the torsional drive.
Embodiment 9. The insertion device according to any one of the preceding embodiments, wherein the torsional drive comprises at least one torsional spring.
Embodiment 10. The insertion device according to any one of the preceding embodiments, wherein the guiding structure is a guiding rail and/or a guiding channel.
Embodiment 11. The insertion device according to any one of the preceding embodiments, wherein a shape of the guiding structure is selected from the group consisting of non-circular, elliptic, spiral-shaped, and triangular.
Embodiment 12. The insertion device according to any one of the preceding embodiments, wherein the shape of the wheel is selected from the group consisting of circular, elliptic, non-circular, triangular, rectangular or polygonal.
Embodiment 13. The insertion device according to any one of the preceding embodiments, wherein the insertion device comprises at least one follower, wherein the guiding structure is configured for guiding the follower.
Embodiment 14. The insertion device according to the preceding embodiment, wherein a shape of the guiding structure is such that friction with the follower is increased in at last one angular position or section.
Embodiment 15. The insertion device according to any one of the two preceding embodiments, wherein the follower is a follower pin, wherein the guiding structure guides the follower pin that is attached to the holding structure.
Embodiment 16. The insertion device according to any one of the preceding embodiments, wherein the insertion device comprises at least one brake element.
Embodiment 17. The insertion device according to any one of the preceding embodiments, wherein the insertion device comprises a friction brake.
Embodiment 18. The insertion device according to any one of the preceding embodiments, wherein the insertion device comprises a medical device locking mechanism configured for preventing the medical device from being pulled back into the housing when the holding structure withdraws the insertion tool from the medical device.
Embodiment 19. The insertion device according to any one of the preceding embodiments, wherein the insertion device comprises an insertion locking mechanism configured for locking and releasing movement of the holding structure, wherein the insertion locking mechanism is configured for interacting with one or more of the wheel, the holding structure guide or the medical device.
Embodiment 20. The insertion device according to any one of the preceding embodiments, wherein the insertion tool comprises at least one insertion cannula or at least one insertion needle.
Embodiment 21. The insertion device according to the preceding embodiment, wherein the insertion device comprises an insertion tool protection mechanism configured for blocked movement of the holding structure from movement towards the proximal position again, or an insertion needle protection mechanism configured for blocked movement of the holding structure from movement towards the proximal position again.
Embodiment 22. The insertion device according to any one of the preceding embodiments, wherein the insertion device comprises at least one protective cap, wherein the protective cap is configured for covering a skin facing opening of the housing.
Embodiment 23. The insertion device according to the preceding embodiment, wherein removal of the protective cap releases and/or unblocks the insertion locking mechanism.
Embodiment 24. The insertion device according to any one of the two preceding embodiments, wherein the torsional drive is pre-tensioned ex-factory or by the user, preferably by pulling off the protective cap which in turn tensions the torsional drive.
Embodiment 25. The insertion device according to any one of the preceding embodiments, wherein one or more of the housing or the protective cap comprise at least one desiccant.
Embodiment 26. The insertion device according to any one of the preceding embodiments, wherein the insertion device comprises at least one user interface, such as a sound generator configured for playing at least one sound when the insertion has been successfully completed.
Embodiment 27. An insertion system comprising at least one medical device and at least one insertion device according to any one of the preceding embodiments for at least partially inserting the medical device into a body tissue of a subject, wherein the medical device is held by the holding structure.
Embodiment 28. The insertion system according to the preceding embodiment, wherein the medical device comprises at least one device selected from the group consisting of: an analyte sensor device; an infusion device; and an electrical stimulating device.
Embodiment 29. The insertion system according to the preceding embodiment, wherein the medical device further comprises at least one electronic remote device configured to communicate and/or control with the medical device.
Embodiment 30. The insertion system according to the preceding embodiment, wherein the electronic remote device is selected from a personal computer, a wearable, a smartphone, a proprietary remote control, a tablet or a server.
Embodiment 31. A method for inserting at least an insertable part of a medical device into a body tissue of a subject, wherein the method comprises using the insertion system according to any one of the preceding embodiments referring to an insertion system, wherein the method comprises the following steps:
a) applying the insertion system to a subject's skin;
b) inserting at least an insertable part of the medical device into the body tissue of the subject wherein the wheel is rotated upon activation by the at least one torsional drive, wherein an axial movement of the insertion tool and the holding structure between the distal position and the proximal position is driven by the wheel, and wherein the guiding structure comprises a shape with an eccentricity relative to a center of the wheel and/or relative to a turning point of the wheel, wherein the eccentricity is unequal to zero, and wherein the shape slows a linear movement around a turning point of the insertion tool.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A, 1B and 1C show an embodiment of a method for inserting at least an insertable part of a medical device using an exemplary embodiment of an insertion system according to the present invention;

FIGS. 2A, 2B, 2C and 2D show embodiments a wheel and guiding structures; and

FIGS. 3A, 3B, 3C, 3D, 3E and 3F show further embodiments of guiding structures.

### DETAILED DESCRIPTION

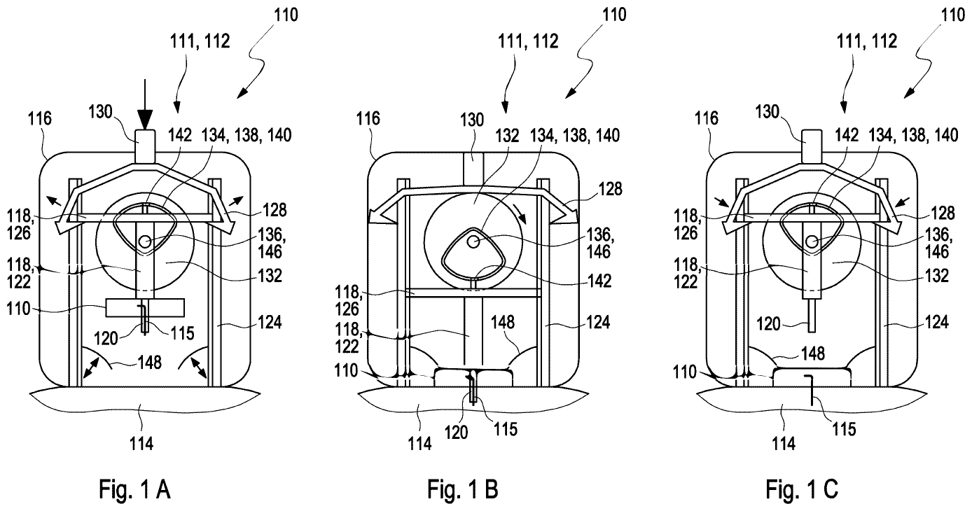

FIGS. 1A to 1C show a sequence of an embodiment of a method for inserting at least an insertable part of a medical device 110 using an exemplary embodiment of an insertion system 111 according to the present disclosure into a body tissue 114. The insertion system 111 comprises at least one insertion device 112 and the medical device 110.

The medical device 110 may be arbitrary element or article being configured for use in the field of medical technology, exemplarily in the field of medical analytics or medical diagnostics. The medical device 110 may be configured for performing at least one medical function and/or for being used in at least one medical process, such as one or more of a therapeutic process, a diagnostic process or another medical process. The medical device 114 may be configured to be mounted on a skin site of an extremity of a subject. The extremity may be selected from the group consisting of: an arm, exemplarily an upper arm; a stomach; a shoulder; a back; hip; a leg. Exemplarily, the extremity may be the upper arm. However, also other applications may be feasible. The medical device 110 may comprise at least one component which may be configured to stay outside of the body tissue 114. Further, the medical device 110 may comprise at least one insertable portion 115 such as an analyte sensor or an infusion cannula or at least one electrical stimulator electrode. The insertable portion may be configured for being inserted into the body tissue 114 of the subject. Exemplarily, the insertion device may be configured for inserting the insertable portion of the medical device 110 into the body tissue 114 of the subject. The medical device 114 may comprise at least one device selected from the group consisting of: an analyte sensor device, e.g. an analyte sensor device configured for detecting at least one analyte in a body fluid of the subject; an infusion device, e.g. a drug infusion device configured for infusing a liquid drug such as insulin or any other liquid drug into a tissue of the subject; and an electrical stimulating device, e.g. configured for stimulating a body tissue of the subject. Such infusion devices include infusion pumps that can be attached to the skin of a patient also known as patch pumps or infusion sets that are connected to an infusion pump often worn in the pocket of a patient and connected to the infusion set by a tubing to deliver the drug from the pump into the skin. Known patch pumps include insulin pumps such as Roche's Accu Chek® Solo Micropump or Terumo's Medisafe patch pump. Known infusion sets, in turn, include Roche's Accu-Chek® Insight Flex or Tandem's Diabetes AutoSoft Infusion set or the infusion sets manufactured by Convatec® such as Neria Guard.

The insertion device 112 may be configured for inserting at least the insertable part of the medical device 112 into the body tissue 114. The insertion device 112 may be configured for transcutaneously or subcutaneously inserting at least the insertable part of the medical device 110 into the body tissue 114, such as by performing an incision or a puncture in a skin of the subject and by transferring the medical device partially into the body tissue. The insertion device 112 may be removed fully or partially after inserting the medical device 112 at least partially into the body tissue 114 of the subject.

The insertion device 112 comprises at least one housing 116 with at least one holding structure 118 adapted to releasably receive the medical device 110. The insertion device 112 further comprises at least one insertion tool 120 configured for inserting at least the insertable part of the medical device 112. The insertion tool 120 can be an insertion cannula. The holding structure 118 may be configured for holding the medical device 118 and/or the holding structure 118 may be configured for holding the insertion tool 120.

The holding structure 118 may be configured for directly or indirectly advancing the holding structure 118 and at least the insertable part of the medical device 110 axially towards the body tissue 114 and for inserting the insertion tool 120 and at least the insertable part of the medical device 110 into the body tissue 114. The holding structure 118 may be configured for directly or indirectly withdrawing the insertion tool 120 from the medical device 110 while leaving at least the inserted part of the medical device 110 in the body tissue 114.

The holding structure 118 may be or may comprise at least one plunger 122 or may be connected to at least one plunger 122 in order to drive the insertion tool 120 to perform a puncture or insertion motion into the body tissue 114 for inserting the medical device 110 and back. The plunger 122 may be configured for directly or indirectly, e.g. via at least one further element, attaching to the medical device 110 and/or the insertion tool 120. The plunger 122 may be configured to directly or indirectly advance the medical device 110 axially towards the skin and to insert the insertion tool, e.g. an insertion cannula, into the skin in a first phase. The plunger 122 may be configured to withdraw the insertion tool 120 from the medical device 110 while leaving the inserted part of the medical device in the skin in a second phase.

The holding structure 118 may comprise at least one holding structure guide, e.g. a guide for the plunger. In the embodiment of FIGS. 1A to 1C, the holding structure guide is embodied as at least one guiding frame 124 and at least one plunger guide 126. The plunger guide 126 may comprise at least one bar. The plunger 122 may be connected to the plunger guide 126. The guiding frame 124 may be configured for guiding the axial movement of the plunger 122. The guiding may comprise directing the motion of the holding structure 118. For example, the housing 116 may comprise the guiding frame 124 configured for guiding an axial movement of the holding structure guide between its distal position and its proximal position.

The insertion device 112 may comprise at least one insertion locking mechanism 128. The insertion locking mechanism 128 may be configured for locking and releasing movement of the holding structure 118. In the embodiment shown in FIGS. 1A to 1C, the insertion locking mechanism 128 may be configured for interacting with the plunger guide 126. For example, advancing the holding structure 118 may be triggered by releasing the insertion locking mechanism 128, e.g. upon actuation of at least one button 130. For example, the insertion locking mechanism 128 may be adapted for preventing an unwanted actuation of the insertion device 112, thereby preventing an unwanted driving of the holding structure 118. The holding structure 118 may have a retracted position or a rest position, in which the holding structure 118 is stored before insertion and in which the insertion tool 120 does not protrude from the insertion device 112. The insertion locking mechanism 128 may be adapted to prevent the holding structure 118 from unwantedly leaving the rest position or a storage position and/or may be adapted to generally prevent an unwanted firing of the insertion device 112. The insertion locking mechanism 128 may comprise at least one locked position (as shown in FIGS. 1A and 1C) and at least one unlocked position (as shown in FIG. 1B), wherein in the locked position, an unwanted firing may be blocked, and wherein in the unlocked position, a firing of the insertion device 112 and, thus, an insertion of the insertable portion 115 of the medical device 110 into the body tissue 114, is enabled. For example, as shown in FIG. 1A, the insertion locking mechanism 128, in a locked position, is adapted to at least partially block movement of the holding structure 118 by engaging with the plunger guide 126. The insertion locking mechanism 128 further may act as a transportation lock or as a transportation safety device, such as for preventing an unwanted firing of the insertion device 112 during transportation or storage. Still, an additional transportation locking device or transportation safety device may be implemented additionally. The insertion locking mechanism 128 may comprise one or more blocking elements, e.g. a plunger locking arm as shown in FIGS. 1A to 1C. The plunger locking arm may comprise at least one hook configured for engaging with the plunger guide 126 in the distal position. Upon pushing of the button 130, the plunger locking arm releases the plunger guide 126, e.g. the button 130 may exert a force on the plunger locking arm such that the hooks move outwards and release the plunger guide 126. In the locked position of the insertion locking mechanism 128 with the blocked holding structure 118 in retracted position shown in FIG. 1C it is also prevented that the user can hurt himself by contacting the insertion tool 120.

The insertion locking mechanism 128 may be operable by hand. For operating the insertion locking mechanism 128, such as triggering releasing the insertion locking mechanism 128, the insertion locking mechanism 128 may comprise at least one operation element, e.g. the button 130. However, other types of operation elements are feasible.

The insertion tool 120 may comprise a tip or a sharp end for inserting the part of the medical device, exemplarily the insertable portion 115 of the medical device 110, into the body tissue 114. The insertion tool 120 may comprise at least one insertion cannula or at least one insertion needle. During insertion, the insertion tool 120 may perform a puncture motion from the distal to the proximal position, thereby creating an incision in the skin, transferring at least the insertable portion 115 of the medical device 110 into the body tissue 114, and, subsequently, a motion into the reverse direction, wherein the insertion tool 120 is pulled out from the body tissue 114, wherein at least the insertable portion 115 of the medical device 110 at least partially remains within the body tissue 114. During insertion, at least the insertable portion 115 of the medical device 110 may fully or partially be surrounded by the insertion tool 120. The holding structure 118, as outlined above, may be or may comprise the plunger 122 which is adapted for pushing the insertion tool 120 axially to perform the puncture motion and which may further be adapted to axially pull back the insertion tool 120 after insertion. After insertion, the insertable part of the medical device 110, exemplarily the insertable portion 115 of the medical device 110, may remain in the body tissue 114 of the subject. The insertion tool 120, however, may be retracted from the body tissue 114 into the insertion device 112 after inserting at least the insertable portion 115 of the medical device 110. The insertion device 112 may comprise an insertion tool protection mechanism configured for blocking movement of the holding structure 118 towards the proximal position again. In the embodiment of FIGS. 1A to 1C, the insertion locking mechanism 128 may function as insertion tool protection mechanism.

The insertion device 112 comprises the at least one wheel 132 comprising a guiding structure 134 configured for guiding an axial movement of the insertion tool 120 and the holding structure 118 between a distal position and a proximal position. The wheel 132 may be configured for exerting a rotational movement about an axis 136 or a center. For example, the shape of the wheel 132 may be selected from the group consisting of circular, elliptic, non-circular, triangular, rectangular or polygonal. The wheel 132 may be a flywheel. The wheel 132 may be a cam or a face cam.

The guiding structure 134 may be at least one two or three dimensional structure or plurality of structures configured for directing the movement, e.g. defining a velocity and/or acceleration of the axial movement, of the insertion tool 120 and the holding structure 118. The guiding structure 134 may be a guiding rail 138 and/or a guiding channel 140. The guiding channel 140 may comprise at least one groove within a surface plane of the wheel 132. The guiding rail 138 may comprise at least one protrusion extending from the surface plane of the wheel 132. The guiding structure 134 comprises a shape with an eccentricity relative to a center of the wheel 132 and/or relative to a turning point of the wheel 132. The eccentricity is unequal to zero.

For example, the shape of the guiding structure 134, e.g. in a plan view, is selected from the group consisting of non-circular, elliptic, spiral-shaped, and triangular. For example, the shape of the guiding structure 134 may be circular. Alternatively, the shape can be configured such that despite an essentially steady angular movement speed of the wheel, the time that the holding structure remains in an extended position, such as the proximal position, is longer than if the guiding structure 134 has a strictly circular form with zero eccentricity. This design is associated with an advantage compared to hitherto known inserters that operate with high velocity insertion and retraction essentially throughout the whole insertion process: the insertion according to the present invention is able to provide more time for the insertable portion 115 of the medical device 110 to penetrate deeper into the skin before the insertion tool 120 is pulled back: during insertion of the insertable portion 115 of the medical device 110 into the skin, due to the inertia of skin the area of the skin adjacent to the insertion point will move away from the medical device 110, in particular if the insertion occurs at a high axial movement velocity. When the speed of axial movement of the insertion tool 120 around its point of closest proximity to the skin is slowed down, there is more time for the depressed part of the skin around the insertion point to relax and move back upwards towards the medical device 110. This way the insertion of the insertable portion 115 of the medical device 110 is improved because the insertable portion 115 will penetrate the skin deeper before the axial retraction movement accelerates again to pull out the insertion tool 120 from the skin while leaving the insertable portion 115 in place.

The insertion device 112, e.g. the wheel 132, may comprise at least one follower 142. The follower may be attached to the holding structure 118. The guiding structure 134 may be configured for guiding the follower 142. The follower may be configured for engaging with the guiding structure 134 and for moving along the guiding structure 134. For example, the follower 142 may be a follower pin, e.g. moving within the guiding channel 140. The follower 142, e.g. the follower pin, may be attached to the holding structure 118. For example, the follower 142 may be a sleigh or wagon moving on the guiding rail 138. The sleigh or wagon may be attached to the holding structure 118. The shape of the guiding structure 134 may be such that friction with the follower is increased in at last one angular position or section.

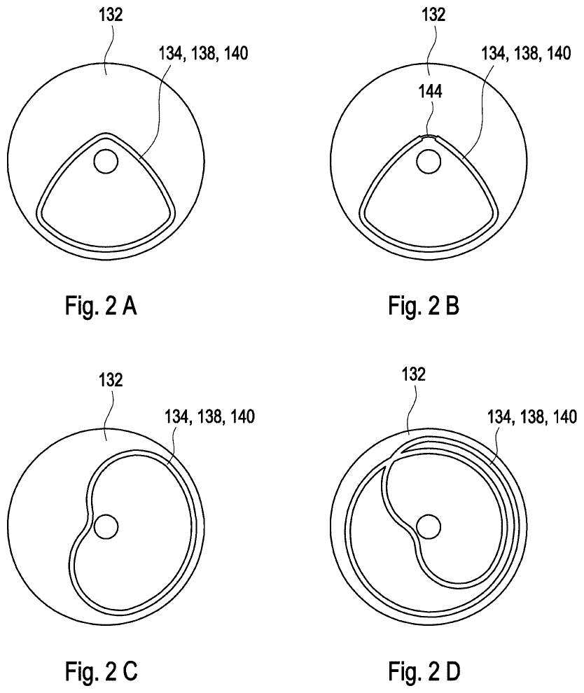

The insertion device 112 comprises at least one brake element 144, e.g. as shown in FIG. 2. In the proximal position of the holding structure 118, e.g. the maximum extended position of the plunger 122, at least one part of the wheel 132 may contact the brake element 144. The brake element 144 may be configured for slowing down the angular movement speed of the wheel 132 for a defined angular braking interval of the wheel 132. Once the end of the braking interval is reached the brake element 144 may cease to inhibit wheel movement and the wheel may accelerate to retract the insertion tool 120. For example, the insertion device 112 may be configured such that the plunger 122 stops movement or moves with very low speed for at least one limited angular interval of wheel movement upon reaching the maximum extended position before initiating the retraction movement. For example, a wheel diameter may be larger in an angular interval where the braking occurs. In this aspect of the wheel 132 an outer margin may contact the brake element 144 which results in slowing down the movement of the wheel. The brake element 134 may comprise a bar, e.g. with a surface of high resistance such as rubber. Additionally or alternatively of extending the diameter of the wheel in a certain angular interval, it may be possible to locally increase the thickness of the wheel such that the brake element contacts the wheel 132 only in the area of increased thickness. Additionally or alternatively, the wheel 132 may comprises the follower pin which extends radially outwards from the wheel 132 and which contacts the brake element 144 such as a leaf spring. Alternatively, the follower pin can be mounted on the side of the wheel 132 and be oriented perpendicular from the circular plane of the wheel 132. The control of the speed of axial movement of the insertion tool 120 and the holding structure 118 by the brake element 144 can also be combined with control provided by the frictional brake.

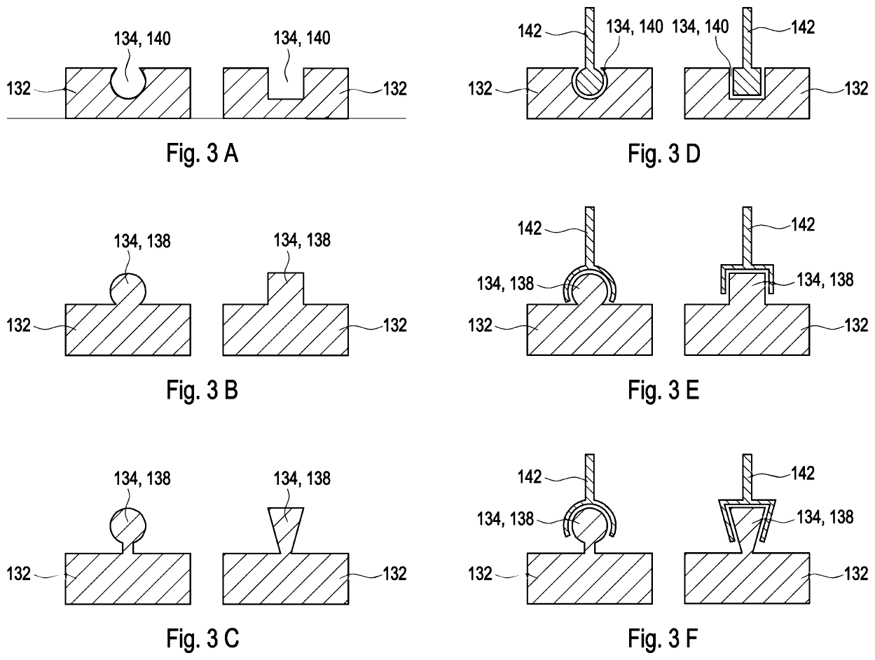

FIGS. 2A to 2D show embodiments a wheel 132 and different embodiments of guiding structures 134. In FIGS. 2A to 2D, the guiding structures 134 are at least partially circular, however, comprise at least one element which deviate from a circular shape. FIGS. 2C and 2D show coils. In principle arbitrary shapes with an eccentricity unequal to zero are possible. Such a shape allows for slowing a linear movement around a turning point of the insertion tool. FIGS. 3A and 3D shows two embodiments of guiding channels 140. In FIG. 3A, on the left, a guiding channel 140 having a circular shape in a cross-sectional view is shown. In FIG. 3A, on the right, a guiding channel 140 having a rectangular shape in a cross-sectional view is shown. In FIG. 3D the corresponding follower 142 is depicted. FIGS. 3B and 3C embodiments of guiding rails 138 are shown, having a circular shape in a cross-sectional view (FIG. 3B, left), a rectangular shape in a cross-sectional view (FIG. 3B, right), a golf tee shape in a cross-sectional view (FIG. 3D, left), and a triangular shape in a cross-sectional view (FIG. 3C, right). FIGS. 3E and F show the corresponding follower 142. Other embodiments and shapes are, however, possible.

As further shown in FIGS. 1A to 1C, the insertion device comprises at least one torsional drive 146 configured for rotating the wheel 132 upon activation. The torsional drive 146 may comprise at least one torsional spring. For example, when loading the torsional spring, the torsional spring is twisted, and can exert, upon activation, a torque in the opposite direction, thereby exerting a rotational force on the wheel 132. The wheel 132 is configured for rotating in response to a rotational force exerted by the torsional drive 146. The torsional drive 146, e.g. the torsional spring, may be preload, e.g. ex-factory. The torsional drive 146, e.g. the torsional spring may be loadable by the user, e.g. the torsional drive may be reloadable.

The insertion device 112 may comprise a medical device locking mechanism 148. The medical device locking mechanism 148, as shown in Figures IA to 1C, may comprise two bendable locking arms configured for preventing the medical device 110 from being pulled back into the housing 116 when the holding structure 118 withdraws the insertion tool 120 from the medical device 110. Movement of the locking arms is indicated with arrows in FIG. 1A.

FIGS. 1A to 1C shows a sequence of method steps of a method which may be performed for inserting at least an insertable part of a medical device 110 into a body tissue 114 of a subject. The method comprises applying the insertion system 111 to a subject's skin. FIG. 1A shows the insertion system 111 applied to the skin. The method further comprises inserting at least an insertable part of the medical device 110 into the body tissue 114 of the subject wherein the wheel 132 is rotated upon activation by the at least one torsional drive 146. An axial movement of the insertion tool 120 and the holding structure 118 between the distal position and the proximal position is driven by the wheel 132. The guiding structure 134 comprises a shape with an eccentricity relative to a center of the wheel 132 and/or relative to a turning point of the wheel 132, wherein the eccentricity is unequal to zero. The shape slows a linear movement around a turning point of the insertion tool 120. FIG. 1B shows the insertion device 112 after activation of the torsional drive 146, wherein movement of the wheel 132 is indicated by an arrow. The method further may comprise retracting the insertion tool 120 from the body tissue 114 of the subject into the insertion device 112 after inserting the medical device 110. FIG. 1C shows the insertion device 112 after retracting the insertion tool 120.

### LIST OF REFERENCE NUMBERS

110 medical device
111 insertion system
112 insertion device
114 body tissue
115 insertable portion
116 housing
118 holding structure
120 insertion tool
122 plunger
124 guiding frame
126 plunger guide
128 insertion locking mechanism
130 button
132 wheel
134 guiding structure
136 Axis
138 guiding rail
140 guiding channel
142 follower
144 brake element
146 torsional drive
148 medical device locking mechanism

While this invention has been described as having an exemplary design, the present invention may be further modified within the spirit and scope of this disclosure. This application is therefore intended to cover any variations, uses, or adaptations of the invention using its general principles.

What is claimed is:

1. An insertion device for inserting at least an insertable part of a medical device into a body tissue of a subject comprising:
at least one housing with at least one holding structure adapted to releasably receive the medical device;
at least one insertion tool configured for inserting at least the insertable part of the medical device;
at least one wheel comprising a guiding structure configured for guiding an axial movement of the insertion tool and the holding structure between a distal position and a proximal position;
at least one torsional drive configured for rotating the wheel upon activation; and
wherein the guiding structure comprises a shape with an eccentricity relative to a center of the wheel and/or relative to a turning point of the wheel, wherein the eccentricity is unequal to zero, and wherein the shape is configured to slow a linear movement around a turning point of the insertion tool.

2. The insertion device according to claim 1, wherein the holding structure is configured for directly or indirectly withdrawing the insertion tool from the medical device while leaving at least the inserted part of the medical device in the body tissue.

3. The insertion device according to claim 1, wherein the holding structure is configured for directly or indirectly advancing the holding structure and at least the insertable part of the medical device axially towards the body tissue and for inserting the insertion tool and at least the insertable part of the medical device into the body tissue.

4. The insertion device according to claim 3, wherein advancing the holding structure is triggered by releasing an insertion locking mechanism.

5. The insertion device according to claim 1, wherein the holding structure comprises at least one holding structure guide, wherein the housing comprises at least one guiding frame configured for guiding an axial movement of the holding structure guide between a distal position and a proximal position.

6. The insertion device according to claim 1, wherein the holding structure comprises at least one plunger.

7. The insertion device according to claim 1, wherein the wheel is configured for rotating in response to a rotational force exerted by the torsional drive.

8. The insertion device according to claim 1, wherein the torsional drive comprises at least one torsional spring.

9. The insertion device according to claim 1, wherein the guiding structure is a guiding rail and/or a guiding channel.

10. The insertion device according to claim 1, wherein a shape of the guiding structure is selected from the group consisting of non-circular, elliptic, spiral-shaped, and triangular.

11. The insertion device according to claim 1, wherein the insertion device comprises at least one follower, wherein the guiding structure is configured for guiding the follower.

12. The insertion device according to claim 1, wherein the insertion device comprises at least one brake element and/or wherein the insertion device comprises a friction brake.

13. An insertion system comprising at least one medical device and at least one insertion device according to claim 1 for at least partially inserting the medical device into a body tissue of a subject, wherein the medical device is held by the holding structure.

14. The insertion system according to claim 13, wherein the medical device comprises at least one device selected from the group consisting of: an analyte sensor device; an infusion device; and an electrical stimulating device.

15. The insertion system according to claim 14, wherein the medical device further comprises at least one electronic remote device configured to communicate and/or control with the medical device.

16. The insertion system according to claim 15, wherein the electronic remote device is selected from a personal computer, a wearable, a smartphone, a proprietary remote control, a tablet or a server.

17. A method for inserting at least an insertable part of a medical device into a body tissue of a subject, wherein the method comprises using the insertion system according to claim 13 and wherein the method further comprises the following steps:
applying the insertion system to a subject's skin;
inserting at least an insertable part of the medical device into the body tissue of the subject, wherein the wheel is rotated upon activation by the at least one torsional drive, wherein an axial movement of the insertion tool and the holding structure between the distal position and the proximal position is driven by the wheel, and wherein the guiding structure comprises a shape with an eccentricity relative to a center of the wheel and/or relative to a turning point of the wheel, wherein the eccentricity is unequal to zero, and wherein the shape slows a linear movement around a turning point of the insertion tool.
